(19) United States
(12) Patent Application Publication
(10) Pub. No.: US 2011/0054613 A1
(43) Pub. Date: Mar. 3, 2011

(54) SPINAL IMPLANT AND METHOD

(75) Inventor: James E. Hansen, Austin, TX (US)

(73) Assignee: MURREN, LLC, Austin, TX (US)

(21) Appl. No.: 12/548,064

(22) Filed: Aug. 26, 2009

(51) Int. Cl. A61F 2/44; A61B 17/86; A61B 17/58

(52) U.S. Cl. 623/17.11; 606/301; 606/86 R

(57) ABSTRACT

A spinal implant includes a tapering box-shaped block made of a bone material; and at least one buttress ridge disposed on at least one surface of the tapering box-shaped block. A spinal implant bolt includes a shaft with a cylindrical body, the shaft having a leading end and a trailing end, wherein the trailing end has at least one slot for receiving a screw driver; and a buttress thread dispose on the shaft in a spiral configuration, wherein the buttress thread has a leading flank facing the leading end of the shaft and a trailing flank facing the trailing end of the shaft, the leading flank forms a smaller angle with a longitudinal axis of the shaft than an angle formed between the trailing flank and the longitudinal axis of the shaft, wherein the spinal implant bolt is made of a bone material.

### BACKGROUND OF INVENTION

1. Field of the Invention

The invention relates generally to spinal implants and methods of using such implants to stabilize and fuse a facet joint.

2. Background Art

The vertebrae in a patient's spinal column are linked to one another by the intervertebral disc and the facet joints. The spinal motion segment includes the intervertebral disc anteriorly and two symmetrical facet joints posteriorly. This three joint complex controls the movement of the vertebrae relative to one another. Each vertebra has a pair of articulating surfaces located on the left side, and a pair of articulating surfaces located on the right side, and each pair includes a superior articular surface and an inferior articular surface. Together the superior and inferior articular surfaces of adjacent vertebra form a facet joint. Facet joints are synovial joints, which means that each joint is surrounded by a capsule of connective tissue and ligments, and produces a fluid to nourish and lubricate the joint. The joint surfaces are coated with cartilage allowing the joints to move or articulate relative to one another, allowing for limited motion of the spinal segments, primarily flexion and extension of the spine.

Diseased, degenerated, impaired, or otherwise painful facet joints and/or discs can require surgery to restore function to the three joint complex. In the lumbar spine, for example, one form of treatment to stabilize the spine and to relieve pain involves the fusion of the facet joint.

One known technique for stabilizing and treating the facet joint involves a trans-facet fusion in which metal pins, screws or bolts penetrate the lamina to fuse the joint. Such a technique has been associated with the risk of further injury to the patient as such trans-lamina facet instrumentation can be difficult to place in a way that it does not damage the spinal canal and/or contact the dura mater of the spinal cord or the nerve root ganglia. Further, trans-facet instrumentation is known to create a rotational distortion, lateral offset, hyper-lordosis, and/or intervertebral foraminal stenosis at the level of instrumentation.

Examples of facet instrumentation currently used to stabilize the lumbar spine include trans-lamina facet screws (“TLFS”) and trans-facet pedicle screws (“TFPS”). TLFS and TFPS implants provide reasonable mechanical stability, but they can be difficult to place, have long trajectories, and surgical access can be confounded by local anatomy. In some instances these implants can result in some degree of foraminal stenosis.

To circumvent some of these problems, spinal implants made of pre-shaped, harvested or synthetic bone, e.g., cortical bone, as a structural fixation have been developed, see for example U.S. Pat. No. 6,485,518 issued to Cornwall et al. These spinal implants may promote natural bone ingrowth resulting in more stable, stronger, and permanent bone fusion.

For example, FIG. 1 shows a cylindrical allograft implant 10 with ridges 12. The facet joint 14 can be identified by using a facet finder 16 followed by drilling to create a socket 18 with a predetermined depth in the facet joint 14. FIG. 2A shows a surgical hammer 20, which can be used to push the allograft implant 10 into the socket 18 in the facet joint 14. FIG. 2B shows that the allograft implant 10 is inserted into the socket 18 in the facet joint 14 by the surgical hammer 20. FIG. 2C-G show 3-D views of two allograft implants 10 inserted inside the facet joints. However, because these methods require drilling cylindrical holes into the facet joint, the integrity of the joint is often compromised causing implantation delay and little surface area for fusion.

Clearly, there is a need for instrumentation and techniques that facilitate the safe and effective stabilization and fusion of facet joints.

### SUMMARY OF INVENTION

One aspect of the invention relates to spinal implants. A spinal implant in accordance with one embodiment of the invention includes a tapering box-shaped block made of a bone material; and at least one buttress ridge disposed on at least one surface of the tapering box-shaped block. The bone material may be cortical bone. The spinal implant may further include a plurality of indentations or perforations to increase its surface areas.

Another aspect of the invention relate to spinal implants. A spinal implant in accordance with one embodiment of the invention includes a tapering box-shaped block made of cancellous bone. The spinal implant may further include a cortical bone cap at its leading end.

Another aspect of the invention relate to spinal implant bolts. A spinal implant bolt in accordance with one embodiment of the invention includes a shaft with a cylindrical body, the shaft having a leading end and a trailing end, wherein the trailing end has at least one slot for receiving a screw driver; and a buttress thread dispose on the shaft in a spiral configuration, wherein the buttress thread has a leading flank facing the leading end of the shaft and a trailing flank facing the trailing end of the shaft, the leading flank forms a smaller angle with a longitudinal axis of the shaft than an angle formed between the trailing flank and the longitudinal axis of the shaft, wherein the spinal implant bolt is made of a bone material.

Other aspects and advantages of the invention will be apparent from the following description and the appended claims.

### BRIEF DESCRIPTION OF DRAWINGS

FIG. 1 shows a prior art of cylindrical allograft implant with ridges.

FIG. 2 shows a prior art of cylindrical allograft implants with ridges and a method of inserting the implants into facet joints

FIG. 3 shows a side view of a spinal implant in accordance with one embodiment of the present invention.

FIG. 4 shows the top view of the spinal implant of FIG. 3.

FIG. 5 shows a perspective view of the spinal implant of FIG. 3.

FIG. 6 shows a side view of a spinal implant in accordance with another embodiment of the present invention.

FIG. 7 shows the top view of the spinal implant of FIG. 6.

FIG. 8 shows a perspective view of the spinal implant of FIG. 6.

FIGS. 9A-9C respectively show the bottom view (9A), a side view (9B), and the top view (9C) of a spinal implant in accordance with yet another embodiment of the present invention.

FIG. 9D shows a top view of a spinal implant bolt in accordance with another embodiment of the invention.

FIG. 9E shows a side view of a spinal implant bolt in accordance with another embodiment of the invention.

FIG. 10 shows an alternative spinal implant similar to that of FIG. 3, in accordance with other embodiment of the invention.

FIG. 11 shows a method for inserting a spinal implant in accordance with one embodiment of the present invention.

FIG. 12 shows a method for inserting a spinal implant in accordance with another embodiment of the present invention.

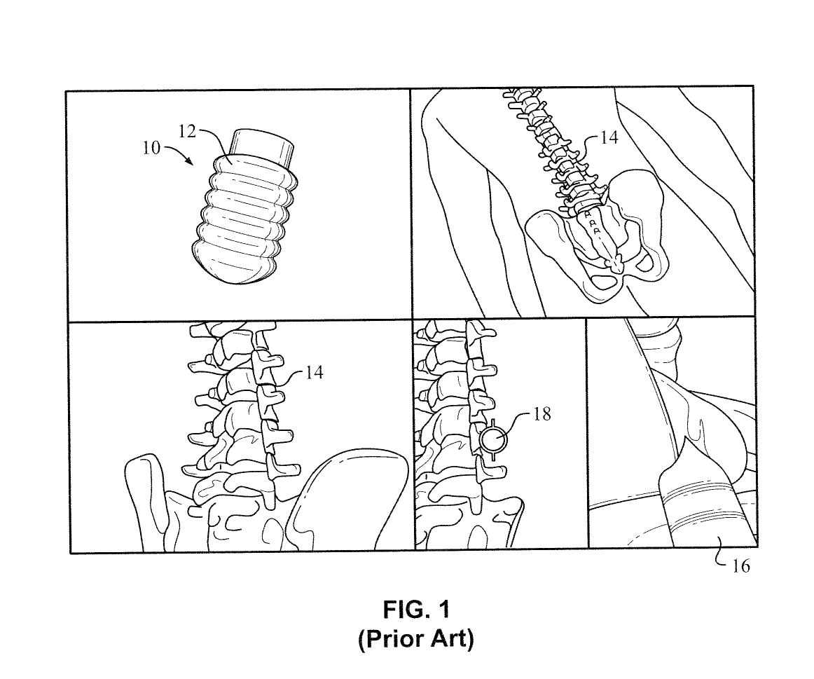

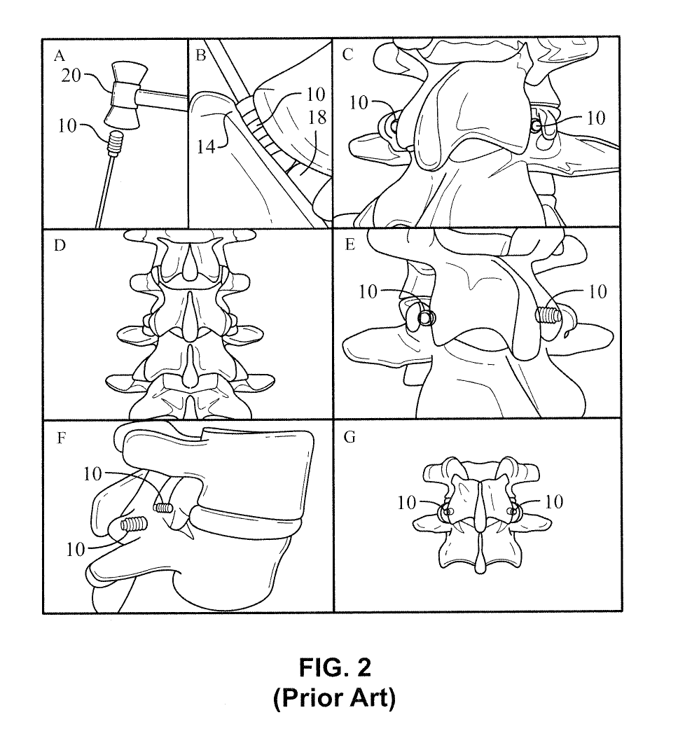

### DETAILED DESCRIPTION

Embodiments of the invention relate to spinal implants and methods of using the spinal implants. Embodiments of the invention include spinal implants (referred to as OsteoFacet Wedge™) having special configurations (e.g., tapering box shape or pyramidal box shape) designed to function as both motion-limiting devices and bone allograft to stabilize and ultimately fuse a joint. By opening the facet capsule and denuding the facet joint of cartilage, the spinal implants of the invention may be forced into a facet joint without drilling a hole into the joint. The implants may then be immobilized and ultimately fused together with the facet joint.

Spinal implants in accordance with embodiments of the invention may also be used as an adjunct to securing non-fusing spinal implants designed to preserve motion in the cervical spine (e.g., artificial cervical disc or artificial cervical nucleus). For example, the spinal implants of the invention may function as an anterior buttresses to the non-fusing spinal implants while passing through the vertebral body to which the spinal implants of the invention may ultimately fuse.

Some embodiments of the present invention have an overall tapering block shape (pyramidal box shape), which may be referred to as “wedges.” In this sense, “wedge” is a descriptive oversimplification of both its shape and function as a spinal implant. A spinal implant “wedge” of the invention may be larger at one end and gradually tapering to a smaller dimension at the other end. A spinal implant wedge of the invention may be used as a motion-limiting device of bone when inserted into a facet joint (such as a lumbar facet joint) to structurally stabilize the facet joint and ultimately create a bone fusion of the joint. These spinal implants may also be used in other parts of the skeletal system or for other purposes, such as in conjunction with a fusion of the anterior intervertebral joint (the disc space).

In addition to having unique shapes, spinal implants of the present invention may be bone allografts, which are made of dense bones (e.g., cortical bone) or less dense (or spongy) bones (e.g., cancellous bone, such as talus (heel bone)). Further, some embodiments of the invention may include both dense and less dense bones. For example, a wedge implant made of cancellous bone may be capped with cortical bone at its leading end to increase its mechanical strength.

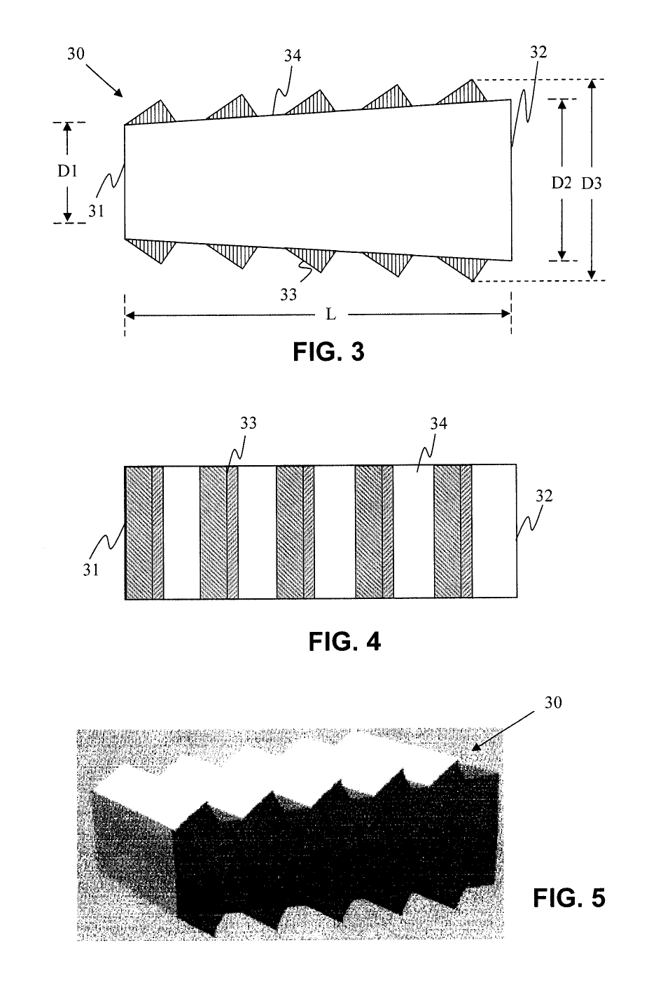

FIG. 3 shows a side view of a spinal implant (a wedge) 30 made of a dense bone (e.g., cortical bone) in accordance with one embodiment of the invention. Such a device may be referred to as OsteoFacet Wedge-Cortical™. FIG. 4 shows a top view of the same device, while FIG. 5 shows a perspective view.

As shown in FIG. 3, the spinal implant 30 comprises a tapering box-shaped block (or wedge-like body) 34 and one or more buttress ridges 33. The spinal implant 30 has a trailing end 32 and a leading end 31. The trailing end 32 (e.g., around 5 mm wide) is slightly larger than the leading end 31 (e.g., around 4 mm wide) to give it a wedge-like (tapering block) shape instead of a regular block shape of conventional facet implant devices. The smaller dimension at the leading end 31 facilitates the placement of the device. This makes it unnecessary to pre-drill a hole in bone for placement of the wedge. For example, such a wedge may be forced into a facet joint.

The dimensions of a spinal implant of the invention, i.e., the length (shown as L in FIG. 3), the dimension of the leading end 31 (shown as 1M in FIG. 3), and the dimension of the trailing end 32 (shown as D2 in FIG. 3), may be selected for the intended application. For example, for use as a facet joint implant, the dimension of the leading end 31, D1, may range from about 3 mm to about 5 mm, preferably around 4 mm. The dimension of the trailing end 32 may be selected in accordance with the dimension of the leading end 31, to provide the desired tapering. For example, the dimension of the trailing end 32, D2, may range from about 4 mm to about 6 mm, preferably about 5 mm.

The length L of a spinal implant 30, i.e., the distance between the trailing end 32 and the leading end 31, may be adjusted for the intended use. For example, for use as a facet joint implant, such lengths L may range from about 7 mm to about 15 mm, preferably between around 9 mm and about 13 mm.

In the example shown in FIG. 5, the trailing end 32 has a square shape, while the leading end 31 has a rectangular shape. That is, the tapering occurs on the top and bottom sides, but not on the left and the right sides of the block. In some embodiments, the tapering may occur on one side, three sides, or four sides. In addition, the shapes of the leading and trailing ends need not be square or rectangular shapes. One skilled in the art would appreciate that other variations and modifications are possible without departing from the scope of the invention.

In addition to the wedge-like shape (tapering box shape), a spinal implant of the invention may include one or more buttress ridges 33 to prevent the “wedge” from sliding back out after placement. As shown in FIG. 3, five buttress ridges 33 are included in this example. One skilled in the art would appreciate that a spinal implant in accordance with embodiments of the invention may have any number of the buttress ridges.

As used herein, a “buttress ridge” refers to a ridge that has a front slope (i.e., the slope of the leading flank) that is less steep than the back slope (the slope of the trailing flank). This configuration will have less resistance when forcing a spinal implant 30 into a facet joint, while providing more resistance to prevent the spinal implant 30 from sliding back out once it is in place. In accordance with some embodiments, the back slope may be 90°, i.e., the trailing flank is perpendicular to the surface of the block on which the buttress ridge is disposed.

The size and shape of each buttress ridge 33 may be varied for the particular application. For example, the spacing between the neighboring ridges may be around 0.75 mm, and the height of each ridge may be around 0.5 mm, for a wedge having the following dimensions: L=9 mm, D1=4 mm, and D2=5 mm (see FIG. 3).

As shown in FIG. 3, a buttress ridge abuts the leading end 31 of the spinal implant 30 to create a wedge shaped front end, the buttress ridges do not cover all the way back to the trailing end 32. As a result, the region near the trailing end 32 has a smaller dimension (D2), as compared to the span of the buttress ridge (D3). This region creates a “counter sink,” which may allow the facet joints to close back over the spinal implant 30 to “capture” the spinal implant 30 in the facet joint after placement.

The particular configuration of spinal implant 30 in FIG. 3 is for illustration only. One of ordinary skill in the art would appreciate that a buttress ridge need not abut the leading end 31 and that a buttress ridge may abut the trailing end 32, if so desired.

FIG. 4 shows the top view of the spinal implant 30 shown in FIG. 3. In this example, the buttress ridges 33 are distributed evenly on the top surface. In addition, the buttress ridges 33 are of the same shape and size. As shown in FIG. 3 and FIG. 5, the buttress ridges 33 may be distributed on both the top an bottom sides of the spinal implant 30. However, in some embodiments, the buttress ridges 33 may be distributed on a single side or more than two sides. One skilled in the art would appreciate that modification and variation of the shape, size, distribution, and the number of the buttress ridges are possible without departing from the scope of the invention.

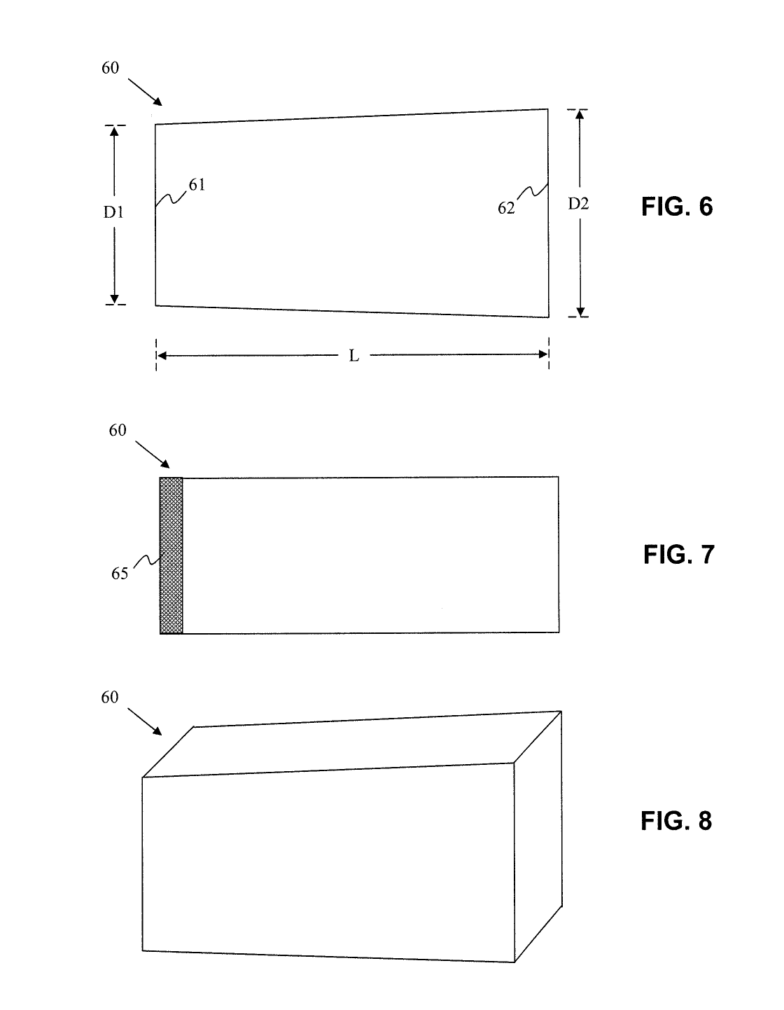

Some embodiments of the invention are designed to encourage more bone growth in the implant. For these embodiments, the implant may be made of a less dense bone, such as cancellous bone. FIG. 6 shows a side view, FIG. 7 shows a top view, and FIG. 8 shows a perspective view of a spinal implant wedge 60, (referred to as OsteoFacet Wedge-Cancellous™) in accordance with one embodiment of the invention.

As shown in FIG. 6, the spinal implant 60 has a tapering block shape (or wedge-like shape). The trailing end 62 is slightly larger than the leading end 61 to give the tapering block shape. Again, the dimensions of the implant 60 may be selected for the desired application. For example, in one example, the dimension (D2) of the trailing end 62 may be about 5 mm wide and tall, and the dimension (D1) of the leading end 61 may be about 4 mm tall (and bout 5 mm wide). The length (L) of the implant 60, i.e., the distance between the trailing end 62 and the leading end 61, may be about 9 mm. This particular embodiment does not have any buttress ridge.

In accordance with some embodiments of the invention, the leading end 61 may be capped with stronger bone materials, such as cortical bone, to protect the cancellous bone of the wedge from damage when inserted into bone joints. Such a “protective” cap (shown as 65 in FIG. 7) may be a thin layer (e.g., 1-2 mm). As a facet joint fixation implant, the spongy structure of the cancellous bones of the spinal implant wedges 60 may help increase the bone fusion rates and aid incorporation of the implants into the facet bones.

FIG. 7 shows a top view of the spinal implant wedge 60 of FIG. 6. The width and the length of the implant in this view may be 5 mm and 9 mm, for example. FIG. 8 shows a perspective view of the spinal implant wedge 60. In this particular example, the tapering occurs on the top sides and the bottom sides. In addition, no buttress ridges are included. One skilled in the art would appreciate that other modifications and variations are possible without departing from the scope of the invention.

In accordance with embodiments of the invention, the spinal implants (e.g., 30 and 60 shown in FIG. 3 and FIG. 6) may be inserted into a facet joint by a press fit of the implants without drilling cylindrical holes or sockets into the joint. Thus, this process may allow the facet joint surface lips to remain intact without compromising the integrity of the facet joint. In addition, as noted above, a “counter sinking” regions near the trailing ends of some spinal implants in accordance with embodiments of the invention may further allow the distracted facet joint surfaces to close back over the implants and “capturing” them inside.

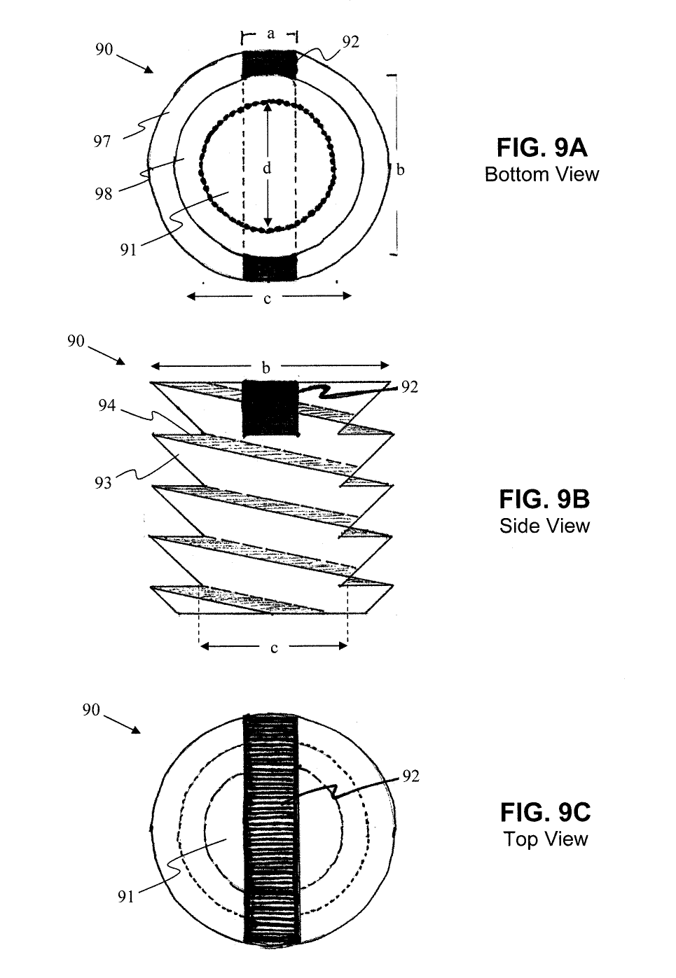

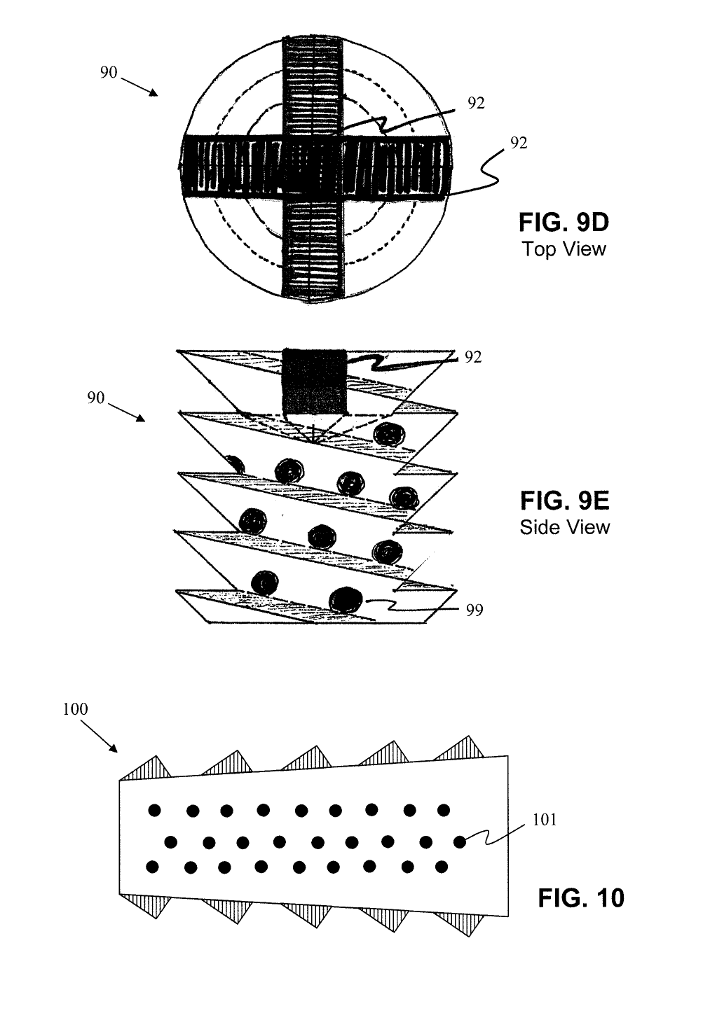

While the wedge-like spinal implants described above may be fitted into a joint, some embodiments of the present invention may include spinal implant bolts (or facet screws), as shown in FIGS. 9A and 9B, that can be screwed into a bone or a joint. Such bolts or screws preferably are made of dense cortical bone (referred to as OsteoFacet Wedge-Cortical Bolt™). The spinal implant bolts may function as threaded bolts, which can be screwed into, for example, a facet joint that has been tapped with matching threads. The size of the spinal implant bolts may be made larger than the afore-mentioned spinal implant wedges, e.g., OsteoFacet Wedge-Cortical™ and OsteoFacet Wedge-Cancellous™. The larger size may help increase its strength, resist backout, and increase stabilization.

Furthermore, in accordance with some embodiments of the present invention, the bolts or screws may be perforated through to create a canal structure, thus, imitating that of a cancellous bone. The resulting perforated structure may allow better incorporation of bone growth into the implant bolts, resulting in stronger structural support than the spinal implant wedges.

FIGS. 9A, 9B, and 9C show the bottom, side, and top views, respectively, of a spinal implant bolt 90 in accordance with one embodiment of the invention. A spinal implant bolt 90 may include a shaft 98, on which a buttress thread 97 is disposed in a spiral configuration as in a traditional bolt or screw. The bolt 90 may include a slot 92 at the trailing end (or top end) of the shaft 98 for applying a screw driver. At the middle of the slot 92, an indentation 91 may be optionally included to facilitate receiving a screw driver. The center channel 91, which may or may not run through the entire length of the shaft or bolt, may encourage the ingrowth of bone tissue, leading to an enhance fusion with the bone. The dimensions of the bolt may be selected for the desired applications. For example, the outside diameters (shown as b) of the bolts 90 may range from about 7 mm to about 15 mm, preferably from about 9 mm to about 13 mm. The diameter of the shaft 98 of the bolt 90 may be about 2 mm smaller than that of the bolt due to the thread depth (which may be about 1 mm). The size of the center channel 91 may be any suitable sizes as long as it does not weaken the bolts too much. For example, in accordance with one embodiment of the invention, the center channel may have a diameter (shown as d) around 5 mm. The slot 92 for receiving a screw driver may be any suitable dimension (shown as a), such as about 2 mm.

Similarly, the length of the bolts, the dimensions and pitches of the threads may be varied for the desired applications. For example, the threads of a bolt may be cut to about 1 mm depth from the outside of the blots, and the pitch of a bolt may be around 1 mm. For example, with an outside diameter (b) of 9 mm, a bolt may have a shaft with a diameter (shown as c) of about 7 mm, i.e., cutting about 1 mm deep threads from the outside diameter.

In accordance with embodiments of the invention, the threads on a bolt are preferably buttress threads that can prevent the screws from backing out after placement. A buttress thread has a leading flank 93 and a trailing flank 94 having different slanting (sloping) surfaces. FIG. 9B shows an example of a buttress thread, in which the leading flank 93 has a more shallow slope relative to the shaft of the screw, while the trailing flank 94 has more steep slope with respect to the shaft of the screws. In preferred embodiments, the surface of the trailing flank 94 may be perpendicular to the longitudinal axis of the screw.

While FIG. 9C shows that the bolt 90 has a single slot 92 for receiving a screw driver, embodiments of the invention may include more than one slots for other types of screw drivers. For example, FIG. 9D shows an alternative embodiment having a cross slots 92 for accepting Philips type screw drivers. The slots 92 may have a depth of about 1-2 mm, depending on the dimensions of the slots. In addition, the slots 92 may gradually slope deeper towards the center to better accommodate a screw driver.

In accordance with some embodiments of the invention, a bolt (or screw) implant 90, may include indentations or perforations (shown as 99 in FIG. 9E) to increase the fusion surface areas. These indentations or perforations 99 may functionally mimic Haversian canals of human bone and allow bone ingrowth to significantly increase the fusion strength. The numbers, locations, and distribution of such indentations or perforations 99 can be selected for the desired effects.

For example, in accordance with one embodiment of the invention, the indentation or perforations 99 may be drilled into the side of the thread, as shown in FIG. 9E. In this particular example, the indentation or perforations 99 are drilled as cylindrical shape of about 1 mm in diameter and about every 45° around the circumference. The perforations may be drilled perpendicular to the axis of the bolts or in other directions.

These perforations may be drilled along the base of buttress threads around the spinal implant bolt 90. Each perforation 99 may be based at the depth of thread trough, flush (or on the same plane) with the perpendicular wall of the buttress thread. Each perforation 99 may be drilled until it reaches 2.5 mm from the center of the vertical axis of the spinal implant bolt 90. In other words, each perforation 99 may have a depth of 2 mm measured from the highest incline wall of the trough. Again, this is only an example, and other modifications or variations are possible without departing from the scope of the invention.

The introduction of indentations or perforations into a spinal implant is not limited to the screw-type implants. The same techniques may be applied to the tapering box-shaped spinal implants, such as those shown in FIGS. 3-8. For example, FIG. 10 shows one example of a spinal implant 100, similar to that of FIGS. 3-5, having indentations or perforations 101 introduced into one or more sides of the spinal implant. Such indentations or perforations would increase the surface areas and encourage bone ingrowth, leading to stronger fusion.

Some embodiments of the invention relate to methods for inserting a spinal implant described above. Various methods for facet joint implant are know in the art. For example, U.S. patent application Ser. No. 12/350,609 (publication No. 2009/0177205) by McCormack et al. discloses methods and apparatus for accessing and treating facet joints. This application is incorporated by reference in its entirety. Such prior art methods may be modified to use a spinal implant (facet fusion implant) of the invention.

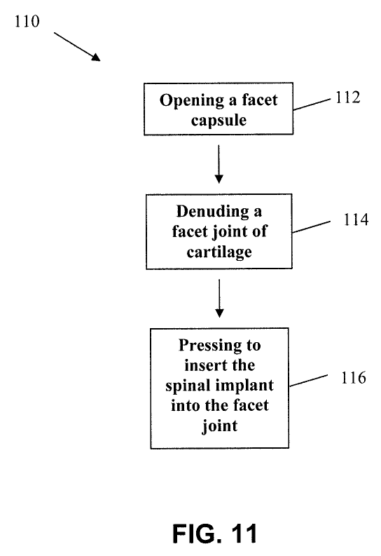

For example, FIG. 11 shows a method 110 for inserting a spinal implant in accordance with one embodiment of the present invention. For example, prior to inserting a spinal implant into a facet joint, the facet capsule may be opened 112 to expose the cartilage of the facet joint by using a suitable surgical tool such as an insertion tool disclosed in the McCormack '609 application. The cartilage of the facet joint may be denuded 114. Then, a spinal implant in accordance with the embodiments of the present invention, e.g., OsteoFacet Wedge-Cortical™ or OsteoFacet Wedge-Cancellous™, may be pressed to insert into the facet joint 116 without drilling. Because a spinal implant of the invention has a wedge-like shape, it can be pressed into a facet joint without first drilling a hole or socket into the joint. In accordance with preferred methods of the invention, the facet joint surface lips may be chipped or removed with a proper tool to remove the fusion-preventing cartilaginous synovial capsule and to expose the underlying cancellous bone for better fusion and incorporation of the implant into the facet joint.

In accordance with some embodiments of the invention, a method of the invention (e.g., method 110 illustrated in FIG. 11) may be performed in conjunction with other procedures, such as that of fusing an anterior intervertebral joint simultaneously or sequentially.

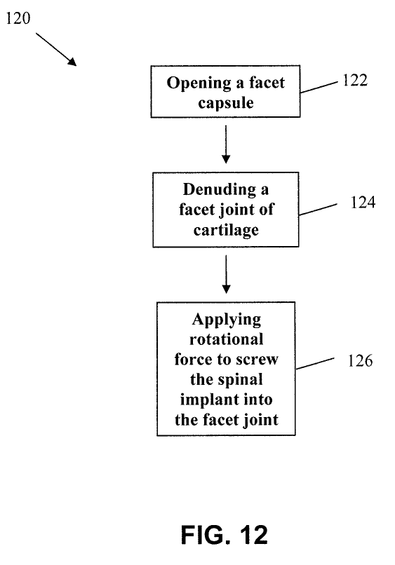

FIG. 12 shows another method 120 for inserting a spinal implant (e.g., a facet screw) in accordance with another embodiment of the present invention. For example, prior to inserting a spinal implant bolt into a facet joint, the facet capsule may be opened 122 to expose the cartilage of the facet joint by using a surgical tool such as a surgical pin. The cartilage of the facet joint may be denuded 124. Then, the spinal implants in accordance with the present invention, e.g., OsteoFacet Wedge-Cortical Bolt™, may be screwed into the facet joint 126 without drilling. In addition, the method 120 may be performed in conjunction with that of fusing an anterior intervertebral joint simultaneously or sequentially.

Advantages of embodiments of the invention may include one or more of the following. The spinal implants of the invention are bone allografts, which will fuse with natural bone and may eventually be absorbed. The tapering box shape (wedge-like shape) of the spinal implants 30 and 60 in accordance with the embodiments of the present invention may allow for implantation without the need for drilling a cylindrical hole into the joint, thus preserving integrity of the joint, improving speed of implantation, and providing more surface area for fusion. The reverse buttress ridges of the spinal implant wedges 30 may provide resistive strength to prevent backout of the implant. Further, the large cortical bolts 90 may provide for increased strength by their size, increased resistance to backout, increased stabilization by their threaded design (screwed into the joint), increased incorporation rate, and increased fusion potential over other cortical implants obtained from the machined channels running through the implant designed for bony ingrowth.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A spinal implant, comprising:
a tapering box-shaped block made of a bone material; and
at least one buttress ridge disposed on at least one surface of the tapering box-shaped block.

2. The spinal implant of claim 1, wherein the bone material is cortical bone.

3. The spinal implant of claim 1, wherein the tapering box-shaped block comprises a plurality of indentations drilled into at least one side.

4. The spinal implant of claim 1, wherein the at least one buttress ridges comprise a plurality of buttress ridges evenly distributed on two opposite sides of the tapering box-shaped block.

5. The spinal implant of claim 4, wherein the two opposite sides of the tapering box-shaped block are not parallel to each other.

6. The implant of claim 1, wherein a leading end of the tapering box-shaped block has a dimension of about 4 mm, a trailing end of the tapering box-shaped block has a dimension of about 5 mm, and a length of the tapering box-shaped block is about 9 mm.

7. The spinal implant of claim 6, wherein a height of each of the at least one buttress ridge is about 0.5 mm.

8. A spinal implant, comprising a tapering box-shaped block made of cancellous bone.

9. The implant of claim 8, wherein the leading end of tapering box-shaped block has a dimension of about 4 mm, a trailing end of tapering box-shaped block has a dimension of about 5 mm, and a length of the tapering box-shaped block is about 9 mm.

10. The spinal implant of claim 8, further comprising a cap made of cortical bone at a leading end of the tapering box-shaped block.

11. The implant of claim 10, wherein the cap is about 1 mm thick.

12. A spinal implant bolt, comprising:
a shaft with a cylindrical body, wherein the shaft has a leading end and a trailing end, wherein the trailing end has at least one slot for receiving a screw driver; and
a buttress thread dispose on the shaft in a spiral configuration, wherein the buttress thread has a leading flank facing the leading end of the shaft and a trailing flank facing the trailing end of the shaft, the leading flank forms a smaller angle with a longitudinal axis of the shaft than an angle formed between the trailing flank and the longitudinal axis of the shaft,
wherein the spinal implant bolt is made of a bone material.

13. The spinal implant bolt of claim 12, further comprising at least one indentation or perforation on the buttress thread.

14. The spinal implant bolt of claim 13, wherein the at least one indentation or perforation is on the leading flank of the buttress thread.

15. The spinal implant bolt of claim 12, wherein the spinal implant bolt is about 9-13 mm long, and the shaft has an outside diameter of about 7 mm.

16. The spinal implant bolt of claim 12, wherein the bone material is cortical bone.

17. The spinal implant bolt of claim 12, wherein the at least one slot comprises two slots in orthogonal configuration for receiving a Phillips type screw driver.

18. A method for placing a spinal implant, comprising:
opening a facet capsule of a facet joint;
denuding the facet joint of cartilage; and
pressing the spinal implant into the facet joint,
wherein the spinal implant comprises:
a tapering box-shaped block made of a bone material; and
at least one buttress ridge disposed on at least one surface of the tapering box-shaped block.

19. A method for placing a spinal implant, comprising:
opening a facet capsule of a facet joint;
denuding the facet joint of cartilage; and
pressing the spinal implant into the facet joint,
wherein the spinal implant comprises a tapering box-shaped block made of cancellous bone.

20. A method for placing a spinal implant bolt, comprising:
opening a facet capsule of a facet joint;
denuding the facet joint of cartilage; and
screwing the spinal implant bolt into the facet joint,
wherein the spinal implant bolt comprises:
a shaft with a cylindrical body wherein the shaft has a leading end and a trailing end, wherein the trailing end has at least one slot for receiving a screw driver; and
a buttress thread dispose on the shaft in a spiral configuration, wherein the buttress thread has a leading flank facing the leading end of the shaft and a trailing flank facing the trailing end of the shaft, the leading flank forms a smaller angle with a longitudinal axis of the shaft than an angle formed between the trailing flank and the longitudinal axis of the shaft,
wherein the spinal implant bolt is made of a bone material.
